SEQUENCE LISTING (49 sequences)

SEQ ID NO: 1 - protein, 101 aa, Artificial Sequence
  amino acid sequence of GvCatcher
MVDKLKEVPTSTKVKFSKKALTENGEDLKGATIQLTKADGSLVKKWVTDGTVTEFELKDGKYTFTETSAPAKYQVATAITFEVKNGKAIVKGIAVTGNTIV

SEQ ID NO: 2 - protein, 99 aa, Artificial Sequence
  amino acid sequence of GbCatcher
VVTDGYSSHDINISKADIDGNEIAGAKIVLTDKAGKQIDSWTSTKELHKVSLKPGTYIFKETLAPEGFEVVTDITFTVNVDGTITVNDKQAKVNNDGVL

SEQ ID NO: 3 - protein, 92 aa, Artificial Sequence
  amino acid sequence of PsCatcher
EKTKVKFSKKALTANGEELKGATIKLTKENGTVIKEWVTDGKLTEFELEDGSYTFTEISAPDKYQVATAITFEVKAGKVLVTGTEVKGNTIV

SEQ ID NO: 4 - protein, 94 aa, Artificial Sequence
  amino acid sequence of SaCatcher
STPSTKVKFSKKALTENGEELKGATIRLTKEDGSLVKEWVTDGTVKEFELKDGKYTFTEISAPDKYQVATAITFEVKNGKVIVKGIEVTGNTIV

SEQ ID NO: 5 - protein, 106 aa, Artificial Sequence
  amino acid sequence of SdCatcher
SGETGQSGNTTIEEDSTTHVKFSKRDINGKELAGAMIELRNLSGQTIQSWVSDGTVKDFYLMPGTYQFVETAAPEGYELAAPITFTIDEKGQIWVDSTLIVGDDPI

SEQ ID NO: 6 - protein, 105 aa, Artificial Sequence
  amino acid sequence of AhCatcher
ITDKETGEDIYLVKDDVTRVSVKKMDITGQKEVAGARLLLKDKEGNVIESWMSTTEARVFEQKLIAGETYTLTEVTAPSGYEVAADITFTVNKDGTVSVDGKAVD

SEQ ID NO: 7 - protein, 100 aa, Artificial Sequence
  amino acid sequence of CpCatcher
NNQKSEVLKLNIIDDLPKEVLFSKHDIAGNELAGATIELSNKADGKVIDTWVSDGQGAHTFNLKPGNYVFTEKAAPEGYEVATAINFTVNPDGTVTSDDV

SEQ ID NO: 8 - protein, 103 aa, Artificial Sequence
  amino acid sequence of RSCatcher
TFTIDKTGKVLVNGEDVNGQVTMYDAALGDVVISKRAVNGTEELEGASLKITDADGKTVAEWVSDSTPKTIQLDAGTYTLTEETAPDGYTVAESIEFVVDAAG

SEQ ID NO: 9 - protein, 110 aa, Artificial Sequence
  amino acid sequence of ScoCatcher
TVEDTSEVQKVEMKDDVTKVQISKTDISGKELPGAKLTILDKDGKTVESWTSEEKPHYIEMLPIGEYTLREETAPDGYLVAEDVKFTVKDTGEIQKVVMKDEVKPTATPT

SEQ ID NO: 10 - protein, 105 aa, Artificial Sequence
  amino acid sequence of SpnCatcher
GNTIVMVDKLKELPPPPSTPPTKVKFSKKALTENGEELKGATIRLTKEDGSLVEEWVTDGTVKEFELKDGKYTFTEISAPAKYQVATAITFEVKNGKAIVKGIEV

SEQ ID NO: 11 - protein, 15 aa, Artificial Sequence
  amino acid sequence of GvTag
NTIVMVDKLKEVPTP

SEQ ID NO: 12 - protein, 13 aa, Artificial Sequence
  amino acid sequence of GbTag
TNIEFVDGNKPHK

SEQ ID NO: 13 - protein, 13 aa, Artificial Sequence
  amino acid sequence of PsTag
NTIVMVDKLKKTP

SEQ ID NO: 14 - protein, 15 aa, Artificial Sequence
  amino acid sequence of SaTag
NTIVMVDKLKELPPP

SEQ ID NO: 15 - protein, 13 aa, Artificial Sequence
  amino acid sequence of SdTag
DPIVMIDNDKPIT

SEQ ID NO: 16 - protein, 13 aa, Artificial Sequence
  amino acid sequence of AhTag
NEIVMKDETTPVG

SEQ ID NO: 17 - protein, 13 aa, Artificial Sequence
  amino acid sequence of CpTag
NHVVMVDGYAPKE

SEQ ID NO: 18 - protein, 13 aa, Artificial Sequence
  amino acid sequence of RSTag
DQIVMVDVAKTTT

SEQ ID NO: 19 - protein, 13 aa, Artificial Sequence
  amino acid sequence of ScoTag
QKVVMKDEVKPTA

SEQ ID NO: 20 - protein, 13 aa, Artificial Sequence
  amino acid sequence of SpnTag
NTIVMVDKLKELP

SEQ ID NO: 21 - protein, 267 aa, Artificial Sequence
  amino acid sequence of the fusion protein GvCatcher-HPF
MVDKLKEVPTSTKVKFSKKALTENGEDLKGATIQLTKADGSLVKKWVTDGTVTEFELKDGKYTFTETSAPAKYQVATAITFEVKNGKAIVKGIAVTGNTIVGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 22 - protein, 265 aa, Artificial Sequence
  amino acid sequence of the fusion protein GbCatcher-HPF
VVTDGYSSHDINISKADIDGNEIAGAKIVLTDKAGKQIDSWTSTKELHKVSLKPGTYIFKETLAPEGFEVVTDITFTVNVDGTITVNDKQAKVNNDGVLGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 23 - protein, 258 aa, Artificial Sequence
  amino acid sequence of the fusion protein PsCatcher-HPF
EKTKVKFSKKALTANGEELKGATIKLTKENGTVIKEWVTDGKLTEFELEDGSYTFTEISAPDKYQVATAITFEVKAGKVLVTGTEVKGNTIVGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 24 - protein, 260 aa, Artificial Sequence
  amino acid sequence of the fusion protein SaCatcher-HPF
STPSTKVKFSKKALTENGEELKGATIRLTKEDGSLVKEWVTDGTVKEFELKDGKYTFTEISAPDKYQVATAITFEVKNGKVIVKGIEVTGNTIVGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 25 - protein, 272 aa, Artificial Sequence
  amino acid sequence of the fusion protein SdCatcher-HPF
SGETGQSGNTTIEEDSTTHVKFSKRDINGKELAGAMIELRNLSGQTIQSWVSDGTVKDFYLMPGTYQFVETAAPEGYELAAPITFTIDEKGQIWVDSTLIVGDDPIGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 26 - protein, 271 aa, Artificial Sequence
  amino acid sequence of the fusion protein AhCatcher-HPF
ITDKETGEDIYLVKDDVTRVSVKKMDITGQKEVAGARLLLKDKEGNVIESWMSTTEARVFEQKLIAGETYTLTEVTAPSGYEVAADITFTVNKDGTVSVDGKAVDGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 27 - protein, 266 aa, Artificial Sequence
  amino acid sequence of the fusion protein CpCatcher-HPF
NNQKSEVLKLNIIDDLPKEVLFSKHDIAGNELAGATIELSNKADGKVIDTWVSDGQGAHTFNLKPGNYVFTEKAAPEGYEVATAINFTVNPDGTVTSDDVGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 28 - protein, 269 aa, Artificial Sequence
  amino acid sequence of the fusion protein RSCatcher-HPF
TFTIDKTGKVLVNGEDVNGQVTMYDAALGDVVISKRAVNGTEELEGASLKITDADGKTVAEWVSDSTPKTIQLDAGTYTLTEETAPDGYTVAESIEFVVDAAGGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 29 - protein, 276 aa, Artificial Sequence
  amino acid sequence of the fusion protein ScoCatcher-HPF
TVEDTSEVQKVEMKDDVTKVQISKTDISGKELPGAKLTILDKDGKTVESWTSEEKPHYIEMLPIGEYTLREETAPDGYLVAEDVKFTVKDTGEIQKVVMKDEVKPTATPTGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 30 - protein, 271 aa, Artificial Sequence
  amino acid sequence of the fusion protein SpnCatcher-HPF
GNTIVMVDKLKELPPPPSTPPTKVKFSKKALTENGEELKGATIRLTKEDGSLVEEWVTDGTVKEFELKDGKYTFTEISAPAKYQVATAITFEVKNGKAIVKGIEVGSGDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 31 - protein, 267 aa, Artificial Sequence; identical to SEQ ID NO: 21
  amino acid sequence of the fusion protein GvTag-GFP

SEQ ID NO: 32 - protein, 265 aa, Artificial Sequence; identical to SEQ ID NO: 22
  amino acid sequence of the fusion protein GbTag-GFP

SEQ ID NO: 33 - protein, 258 aa, Artificial Sequence; identical to SEQ ID NO: 23
  amino acid sequence of the fusion protein PsTag-GFP

SEQ ID NO: 34 - protein, 260 aa, Artificial Sequence; identical to SEQ ID NO: 24
  amino acid sequence of the fusion protein SaTag-GFP

SEQ ID NO: 35 - protein, 272 aa, Artificial Sequence; identical to SEQ ID NO: 25
  amino acid sequence of the fusion protein SdTag-GFP

SEQ ID NO: 36 - protein, 271 aa, Artificial Sequence; identical to SEQ ID NO: 26
  amino acid sequence of the fusion protein AhTag-GFP

SEQ ID NO: 37 - protein, 266 aa, Artificial Sequence; identical to SEQ ID NO: 27
  amino acid sequence of the fusion protein CpTag-GFP

SEQ ID NO: 38 - protein, 269 aa, Artificial Sequence; identical to SEQ ID NO: 28
  amino acid sequence of the fusion protein RSTag-GFP

SEQ ID NO: 39 - protein, 276 aa, Artificial Sequence; identical to SEQ ID NO: 29
  amino acid sequence of the fusion protein ScoTag-GFP

SEQ ID NO: 40 - protein, 271 aa, Artificial Sequence; identical to SEQ ID NO: 30
  amino acid sequence of the fusion protein SpnTag-GFP

SEQ ID NO: 41 - protein, 278 aa, Artificial Sequence
  amino acid sequence of the fusion protein SP-GvTag-RBD-His
MGILPSPGMPALLSLVSLLSVLLMGCVAGSGNTIVMVDKLKEVPTPGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

SEQ ID NO: 42 - protein, 276 aa, Artificial Sequence
  amino acid sequence of the fusion protein SP-PsTag-RBD-His
MGILPSPGMPALLSLVSLLSVLLMGCVAGSGNTIVMVDKLKKTPGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

SEQ ID NO: 43 - protein, 278 aa, Artificial Sequence
  amino acid sequence of the fusion protein SP-SaTag-RBD-His
MGILPSPGMPALLSLVSLLSVLLMGCVAGSGNTIVMVDKLKELPPPGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

SEQ ID NO: 44 - protein, 18 aa, Artificial Sequence
  amino acid sequence of the peptide tag GvTag001
RVGNTIVMVDKLKEVPTP

SEQ ID NO: 45 - protein, 19 aa, Artificial Sequence
  amino acid sequence of the peptide tag GvTag002
KKVGNTIVMVDKLKEVPTP

SEQ ID NO: 46 - protein, 18 aa, Artificial Sequence
  amino acid sequence of the peptide tag GvTagOpti
KVGNTIVMVDKLKEVPTP

SEQ ID NO: 47 - protein, 281 aa, Artificial Sequence
  amino acid sequence of the fusion protein Sp-GvTag001-RBD-His
MGILPSPGMPALLSLVSLLSVLLMGCVAGSGRVGNTIVMVDKLKEVPTPGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

SEQ ID NO: 48 - protein, 282 aa, Artificial Sequence
  amino acid sequence of the fusion protein Sp-GvTag002-RBD-His
MGILPSPGMPALLSLVSLLSVLLMGCVAGSGKKVGNTIVMVDKLKEVPTPGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

SEQ ID NO: 49 - protein, 281 aa, Artificial Sequence
  amino acid sequence of the fusion protein Sp-GvTagOpti-RBD-His
MGILPSPGMPALLSLVSLLSVLLMGCVAGSGKVGNTIVMVDKLKEVPTPGSGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

(19) United States
(12) Patent Application Publication
(10) Pub. No.: US 2024/0228541 A9
(43) Pub. Date: Jul. 11, 2024

(54) COMBINATION OF PEPTIDE LINKERS FOR PROTEIN COVALENT SELF-ASSEMBLY USING SPONTANEOUS ISOPEPTIDE BOND

(71) Applicant: SUN YAT-SEN UNIVERSITY, Guangdong (CN)

(72) Inventors: Hui ZHANG, Guangdong (CN); Xiantao ZHANG, Guangdong (CN)

(73) Assignee: SUN YAT-SEN UNIVERSITY, Guangdong (CN)

(21) Appl. No.: 18/556,677

(22) PCT Filed: Mar. 28, 2022
(86) PCT No.: PCT/CN2022/083430

§ 371 Date: Oct. 23, 2023

(30) Foreign Application Priority Data
Apr. 23, 2021 (CN) 202110444350.7
Jul. 31, 2021 (CN) 202110876301.0

(51) Int. Cl. C07K 7/08; C12N 15/63

(52) U.S. Cl. CPC C07K 7/08; C12N 15/63

(57) ABSTRACT

Disclosed is a combination of peptide linkers for protein covalent self-assembly using a spontaneous isopeptide bond, including a peptide linker 1 and a peptide linker 2. The peptide linker 1 contains a peptide chain having an amino acid sequence as shown in SEQ ID NO: 5, and the peptide linker 2 contains any one or more of peptide chains having amino acid sequences as shown in SEQ ID NOs: 11, 13, 14, and 46. The present disclosure constructs a novel protein self-assembly system based on an isopeptide bond, which effectively solves the problem of low binding efficiency of an existing SpyTag/SpyCatcher system, can significantly improve the reaction efficiency of molecules or components fused to a peptide tag and polypeptide thereof, and has a binding efficiency stronger than that of a SpyTag003/SpyCatcher003 system.

### 1. TECHNICAL FIELD

The present disclosure relates the technical field of protein self-assembly, in particular to, a combination of peptide linkers for protein covalent self-assembly using a spontaneous isopeptide bond.

### 2. BACKGROUND ART

Cell function depends upon the interaction of a large number of non-covalent proteins-proteins; and the precise arrangement of proteins in a complex influences and determines their functions. Non-covalent interaction is always relatively weak, and even will be usually interrupted under the conditions such as, long period or some mechanical force. Therefore, the ability to design the interaction of covalent proteins-proteins can bring a series of new opportunities for Basic Research, Synthetic Biology, and Biotechnology.

The interaction of covalent proteins is usually mediated by a disulfide bond; but disulfide bond is reversible and not suitable for the reduction of cellular space, and even may disturb protein folding. Due to small molecular weight, peptide tag minimizes its interference on protein functions and thus, it is a common tool to analyze and modify a protein. Gene coding is conducted very easily via a peptide tag; due to its small size, biosynthesis costs and immunogenic interference introduced will be reduced. However, there is a little of higher affinity in the interaction between a peptide tag and a protein, which thus limits its effect in the formation of a stable complex.

Currently, the protein capable of spontaneously forming an isopeptide bond has been always applied to develop a peptide tag, and is covalently bound to the corresponding binding polypeptide thereof (i.e., a linker of the two parts) mutually, thus providing irreversible interaction. The protein capable of spontaneously forming an isopeptide bond can be expressed as two fragments: a peptide tag and a polypeptide binding partner. These two fragments can achieve covalent recombination via the formation of an isopeptide bond, such that molecules or components connected to the pepetide tag and the polypeptide binding partner, respectively are fused.

Isopeptide bond is an amido bond formed between side-chain carboxyl groups or side-chain amino groups. In the case of typical biological conditions, such a bond is chemically nonreversible and is resistant to most of proteases. Isopeptide bond is covalent chemically and thus able to produce ultra-strong protein binding effects. On the condition that noncovalent interaction will be dissociated rapidly, e.g., high temperature, high pressure, rigorous chemical treatment or long period, the isopeptide bond formed between a peptide tag and its polypeptide binding partner can still achieve stable binding.

There exist some proteins having very strong interaction in some Gram-positive bacteria. For example, the fibronectin-binding protein (FbaB) of Streptococcus pyogenes applied to the fields of therapeutics, biological materials and vaccines contains an adhesin domain (CnaB2); the 31st lysine (K) and the 117th aspartic acid (D) in the domain can spontaneously form an isopeptide bond under the catalysis of the 77th glutamic acid (E). In the previous scientific researches, the CnaB2 of Streptococcus pyogenes FbaB protein have been divided into a SpyTag peptide having 13 residues and a SpyCatcher peptide having 116 residues to establish a Spy Tag/SpyCatcher system, thus achieving gene coding and covalent interaction between peptides and proteins. The SpyTag/SpyCatcher system provides a simple, specific and genetic-coding method to create various biological materials, for use in multiple aspects, including biological materials, next generation sequencing (NGS), enzyme stabilization, vaccine development, etc.

Even though the SpyTag/SpyCatcher technology has been applied to multiple aspects, the reaction speed and efficiency are still lower. To improve such a situation, on the one hand, an existing SpyTag/SpyCatcher sequence, e.g., a SpyTag003/SpyCatcher003 system is modified and optimized. On the other hand, a protein capable of spontaneously forming an isopeptide bond in other bacteria can be sought, so as to establish a more excellent linking system.

### SUMMARY OF THE INVENTION

The objective of the present disclosure is to overcome the above shortcomings in the prior art, and to provide a combination of peptide linkers for protein covalent self-assembly using a spontaneous isopeptide bond.

The first objective of the present disclosure is to provide a combination of peptide linkers for protein covalent self-assembly using a spontaneous isopeptide bond.

A second objective of the present disclosure is to provide a recombinant and synthetic peptide chain.

A third objective of the present disclosure is to provide a nucleic acid molecule.

A fourth objective of the present disclosure is to provide a vector.

A fifth objective of the present disclosure is to provide a cell.

A sixth objective of the present disclosure is to provide use of any of the combination in self-assembling two molecules or components using an isopeptide bond.

A seventh objective of the present disclosure is to provide a method for self-assembling two molecules or components using an isopeptide bond.

An eighth objective of the present disclosure is to provide a kit for self-assembling two molecules or components using an isopeptide bond.

A ninth objective of the present disclosure is to provide use of one or more of the combination, the nucleic acid molecule, the vector, or the cell in preparing a kit for self-assembling two molecules or components, or in self-assembling two molecules or components using an isopeptide bond.

To achieve the above objectives, the present disclosure is achieved through the following technical solution:

In the present disclosure, GvTag/SdCatcher (SEQ ID NO: 11 and SEQ ID NO: 5), PsTag/SdCatcher (SEQ ID NO: 13 and SEQ ID NO: 5), and SaTag/SdCatcher (SEQ ID NO: 14 and SEQ ID NO: 5) are applied to establish a combined polypeptide-peptide tag linking system capable of spontaneously forming an isopeptide bond. Further, corresponding amino acids are modified and optimized at the N terminal of the peptide tag GvTag such that the binding efficiency is stronger than that of the SpyTag003/SpyCatcher003 system.

Specifically, the other 10 bacteria having a CnaB2 adhesin domain sequence are found via alignment with the CnaB2 sequence of Streptococcus pyogenes. These bacteria may include Gardnerella vaginalis (Gv), Granulicatella balaenopterae (Gb), Peptostreptococcus sp (Ps), Streptococcus anginosus subsp (Sa), Streptococcus dysgalactiae (Sd), Anaerobutyricum hallii (Ah), Clostridium perfringens (Cp), Ruminococcus sp (Rs), Streptococcus constellatus (Sco), and Streptococcus pneumoniae (Spn).

In the present disclosure, to obtain an isopeptide bond system with higher binding efficiency, the corresponding CnaB2 is decomposed into a Tag peptide and a Catcher protein, to establish 10 different Tag-GFP clones and 10 different Catcher-HPF clones, and then these clones are expressed and purified in an E. coli system. Based on the yield of the expression proteins, Catcher proteins of Peptostreptococcus sp, Streptococcus anginosus subsp, and Streptococcus dysgalactiae (PsCatcher, SaCatcher, and SdCatcher) are picked out and incubated with the Tag-GFP of the 10 bacteria above in a way of permutation and combination. Protein electrophoresis and Coomassie brilliant blue are applied to screen out three combinations with more efficient binding capacity, e.g., the combination of SdCatcher and GvTag-GFP, the combination of SdCatcher and PsTag-GFP, and the combination of SdCatcher and SaTag-GFP.

These three forms of combinations are then applied to a Receptor Binding Domain (RBD) subunit nanovaccine system of the S protein of SARS-CoV-2 novel coronavirus. Results show that GvTag-RBD and SdCatcher-HP are self-assembled to a spherical tetracosemer nanoparticle. The use of GvTag/SdCatcher, PsTag/SdCatcher, and SaTag/SdCatcher has effectively improved binding efficiency compared to the existing Spy Tag/SpyCatcher system. Moreover, the protein binding efficiency is optimal when using the GvTag/SdCatcher system. The GvTag sequence is further optimized to obtain a GvTagOpti/SdCatcher system which may further improve the binding efficiency.

Mice are immunized with the RBD-ferritin subunit tetracosemer antigen nanoparticle obtained from the linking systems GvTagOpti/SdCatcher, PsTag/SdCatcher, and SaTag/SdCatcher, which may overcome the shortcoming of insufficient immunogenicity of the RBD monomer, effectively trigger stronger immunoreaction, and improve antibody titer. The linking systems GvTagOpti/SdCatcher, PsTag/SdCatcher, and SaTag/SdCatcher of the present disclosure can significantly improve the reaction efficiency of molecules or components fused to a peptide tag and polypeptide thereof, and has a simple preparation method and a high protein expression level.

Therefore, the present disclosure sets forth the following features:

A combination of peptide linkers for protein covalent self-assembly using a spontaneous isopeptide bond, including a peptide linker 1 (a polypeptide) and a peptide linker 2 (a peptide tag); the peptide linker 1 contains a peptide chain having an amino acid sequence as shown in SEQ ID NO: 5, and the peptide linker 2 contains any one or more of peptide chains having amino acid sequences as shown in SEQ ID NO: 11, SEQ ID NO: 13, and SEQ ID NO: 14.

SEQ ID NO: 5:
SGETGQSGNTTIEEDSTTHVKFSKRDINGKELAGAMIELRNLSGQTIQS
WVSDGTVKDFYLMPGTYQFVETAAPEGYELAAPITFTIDEKGQIWVDST
LIVGDDPI;
SEQ ID NO: 11:
NTIVMVDKLKEVPTP;
SEQ ID NO: 13:
NTIVMVDKLKKTP;
SEQ ID NO: 14:
NTIVMVDKLKELPPP.

Preferably, the peptide linker 2 contains a peptide chain having an amino acid sequence as shown in SEQ ID NO: 11.

More preferably, the peptide linker 2 contains a peptide chain having an amino acid sequence as shown in SEQ ID NO: 46 (the peptide chain as shown in SEQ ID NO: 11 further has three KVG amino acids at the N terminal).

SEQ ID NO: 46:
KVGNTIVMVDKLKEVPTP.

The present disclosure further sets forth a recombinant and synthetic peptide chain, including one or more of the peptide linker 1 and/or the peptide linker 2 in the combination according to any one of claim 1 or claim 2.

The present disclosure further sets forth a nucleic acid molecule, including one or more nucleotide sequences encoding the peptide linker 1 and/or the peptide linker 2 in any one of the combinations.

The present disclosure further sets forth a vector, including the nucleic acid molecule.

The present disclosure further sets forth a cell, including the nucleic acid molecule or the vector.

The present disclosure further sets forth use of any of the combination in self-assembling two molecules or components using an isopeptide bond; the two molecules or components are connected with the peptide linker 1 (polypeptide) and the peptide linker 2 (peptide tag) in the combination, respectively.

Further, the present disclosure sets forth a method for self-assembling two molecules or components using an isopeptide bond, including:
providing a first molecule or a first component containing the peptide linker 1 in the combination according to claim 1 or claim 2;
providing a second molecule or a second component containing the peptide linker 2 in the combination according to claim 1 or claim 2; and
contacting the first molecule or the first component with the second molecule or the second component in the case of spontaneously forming an isopeptide bond between the peptide linker 1 and the peptide linker 2, such that the first molecule or the first component is self-assembled with the second molecule or the second component to form a complex using the isopeptide bond.

Furthermore, the present disclosure sets forth a kit for self-assembling two molecules or components using an isopeptide bond, including: one of the peptide linker 1 (polypeptide) and the peptide linker 2 (peptide tag) in the combination, the nucleic acid molecule encoding the peptide linker 1 and the peptide linker 2 in the combination, and a cell containing a vector encoding the peptide linker 1 and the peptide linker 2 in the combination.

The present disclosure further sets forth use of one or more of the combination, the nucleic acid molecule, the vector, and the cell in preparing a kit for self-assembling two molecules or components, or in self-assembling two molecules or components using an isopeptide bond, falling within the protection scope of the present disclosure.

Compared with the prior art, the present disclosure has the following beneficial effects:

The present disclosure constructs novel protein self-assembly systems GvTag/SdCatcher, PsTag/SdCatcher, and SaTag/SdCatcher based on an isopeptide bond, which effectively solves the problem of low binding efficiency of an existing SpyTag/SpyCatcher system, can significantly improve the reaction efficiency of molecules or components fused to a peptide tag and polypeptide thereof, and has a binding efficiency stronger than that of a Spy Tag003/SpyCatcher003 system. Moreover, the GvTagOpti/SdCatcher system, the PsTag/SdCatcher system, and the SaTag/SdCatcher system of the present disclosure have a simple preparation method and a high protein expression level.

### BRIEF DESCRIPTION OF THE DRAWINGS

FIG. 1 shows sequence alignment of Catcher polypeptides from different bacteria

with SpyCatcher;

FIG. 2 shows sequence alignment of Tag peptides from different bacteria with Spy Tag;

FIG. 3 is a schematic diagram showing structures of fusion proteins Tag-GFP, Catcher-HPF, and Tag-RBD;

FIG. 4 is a structure diagram of a plasmid expressing SdCatcher-HPF;

FIG. 5 is a structure diagram of a plasmid expressing GvTag-GFP;

FIG. 6 shows Coomassie Blue staining and protein yield of fusion proteins Tag-GFP and Catcher-HPF from different bacteria;

FIG. 7 shows Coomassie Blue staining of binding proteins obtained after the Catcher-HPF proteins from Peptostreptococcus sp (Ps), Streptococcus anginosus subsp (Sa), and Streptococcus dysgalactiae (Sd) are incubated with Tag-GFP from 10 bacteria in a way of permutation and combination.

FIG. 8 is a structure diagram showing a plasmid expressing GvTag-RBD;

FIG. 9 is a diagram of Coomassie Blue staining showing linking efficiency of binding proteins obtained after three combined linking systems GvTag/SdCatcher, PsTag/SdCatcher and SaTag/SdCatcher, as well as linking systems SpyTag/SpyCatcher and Spy Tag003/SpyCatcher003 are applied to an RBD nanovaccine system of novel coronavirus;

FIG. 10 is a diagram of Coomassie Blue staining showing linking efficiency of binding proteins of the GvTag/SdCatcher, GvTag001/SdCatcher, GvTag002/SdCatcher, and GvTagOpti/SdCatcher systems;

FIG. 11 is a diagram of Coomassie Blue staining showing protein linking efficiency of the GvTagOpti/SdCatcher and Spy Tag003/SpyCatcher003systems;

FIG. 12 shows purification of RBD-HPF proteins linked using an isopeptide bond of the GvTagOpti/SdCatcher system, the PsTag/SdCatcher system, and the SaTag/SdCatcher system by molecular sieve; and

FIG. 13 shows determination of valence of serum RBD antibody after two weeks that mice are immunized with the RBD-HPF proteins linked using an isopeptide bond of the GvTagOpti/SdCatcher system, the PsTag/SdCatcher system, the SaTag/SdCatcher system, and the SpyTag003/SpyCatcher003 system.

### DETAILED DESCRIPTION OF THE EMBODIMENTS

The present disclosure is described in detail below with reference to the accompanying drawings and specific examples. The examples are merely used to explain the present disclosure, but not construed as limiting the scope of the present disclosure. The test methods used in the following examples are conventional methods unless otherwise specified; the materials and reagents used are commercially available reagents and materials unless otherwise specified.

### EXAMPLE 1 OBTAINING OF BACTERIA HAVING A SEQUENCE OF A CNAB2 ADHESIN DOMAIN

### I. Experimental Method

Bacteria having a sequence of a CnaB2 adhesin domain were found via alignment

with the CnaB2 sequence of Streptococcus pyogenes.

### II. Experimental Results

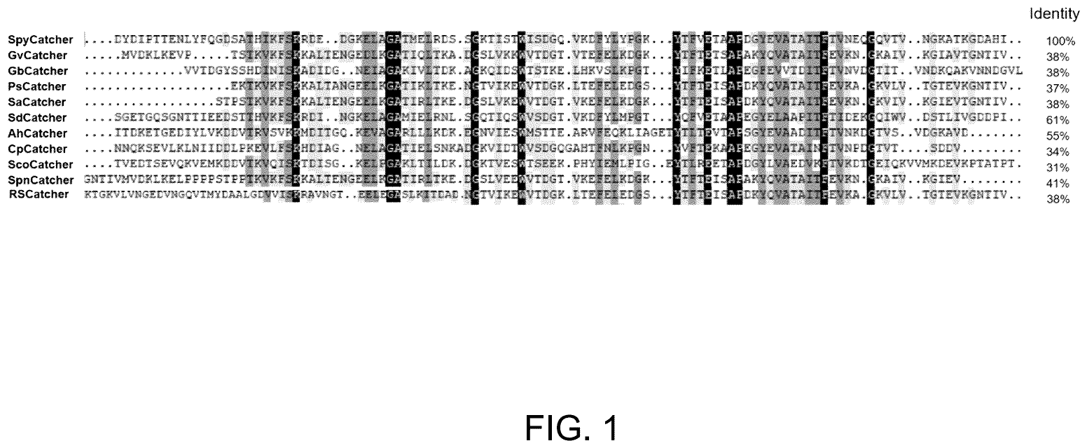

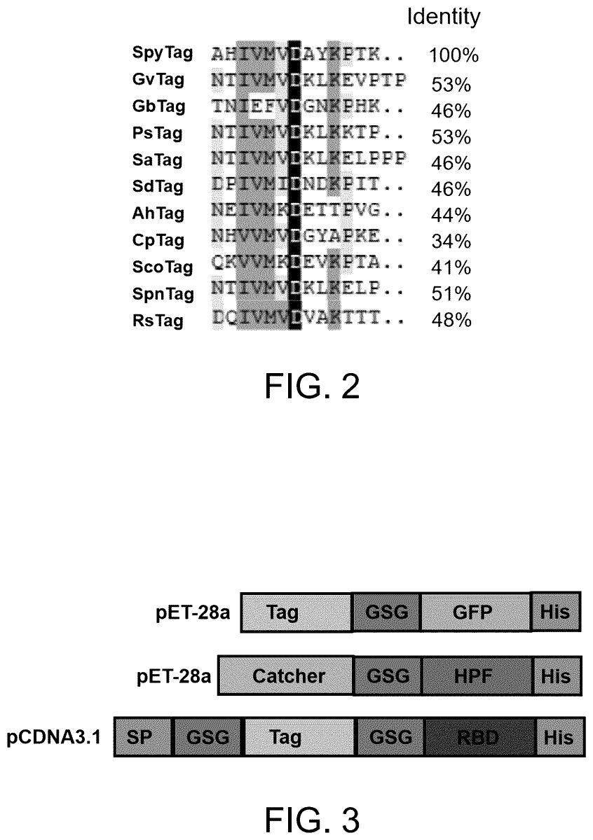

The sequence alignment of the Catcher polypeptides from different bacteria with SpyCatcher, the sequence alignment of the Tag peptide with Spytag, as well as structure diagrams of the fusion proteins Tag-GFP and Catcher-HPF are shown in FIGS. 1-2, respectively. The other 10 bacteria having a sequence of a CnaB2 adhesin domain were found via alignment with the CnaB2 sequence of Streptococcus pyogenes. These bacteria may include Gardnerella vaginalis (Gv), Granulicatella balaenopterae (Gb), Peptostreptococcus sp (Ps), Streptococcus anginosus subsp (Sa), Streptococcus dysgalactiae (Sd), Anaerobutyricum hallii (Ah), Clostridium perfringens (Cp), Ruminococcus sp (Rs), Streptococcus constellatus (Sco), and Streptococcus pneumoniae (Spn).

### EXAMPLE 2 CONSTRUCTION OF NOVEL CATCHER-HPF AND TAG-GFP FUSION PROTEINS, SCREENING OF A COMBINATION WITH HIGH PROTEIN-BINDING EFFICIENCY

### I. Experimental Method

1. Separation of the sequence of a CnaB2 adhesin domain

10 Catcher polypeptide sequences (amino acid sequences are shown in SEQ ID NOs: 1-10, respectively) and 10 Tag peptides (amino acid sequences are shown in SEQ ID NOs: 11-20, respectively) from 10 bacteria, e.g., Gv, Gb, Ps, Sa, Sd, Ah, Cp, Rs, Sco, and Spn.

(amino acid sequence of GvCatcher)
SEQ ID NO: 1
MVDKLKEVPTSTKVKFSKKALTENGEDLKGATIQLTKADGSLVKKWVTD
GTVTEFELKDGKYTFTETSAPAKYQVATAITFEVKNGKAIVKGIAVTGN
TIV;
(amino acid sequence of GbCatcher)
SEQ ID NO: 2
VVTDGYSSHDINISKADIDGNEIAGAKIVLTDKAGKQIDSWTSTKELHK
VSLKPGTYIFKETLAPEGFEVVTDITFTVNVDGTITVNDKQAKVNNDGV
L;
(amino acid sequence of PsCatcher)
SEQ ID NO: 3
EKTKVKFSKKALTANGEELKGATIKLTKENGTVIKEWVTDGKLTEFELE
DGSYTFTEISAPDKYQVATAITFEVKAGKVLVTGTEVKGNTIV;
(amino acid sequence of SaCatcher)
SEQ ID NO: 4
STPSTKVKFSKKALTENGEELKGATIRLTKEDGSLVKEWVTDGTVKEFE
LKDGKYTFTEISAPDKYQVATAITFEVKNGKVIVKGIEVTGNTIV;
(amino acid sequence of SdCatcher);
SEQ ID NO: 5
SGETGQSGNTTIEEDSTTHVKFSKRDINGKELAGAMIELRNLSGQTIQS
WVSDGTVKDFYLMPGTYQFVETAAPEGYELAAPITFTIDEKGQIWVDST
LIVGDDPI;
(amino acid sequence of AhCatcher)
SEQ ID NO: 6
ITDKETGEDIYLVKDDVTRVSVKKMDITGQKEVAGARLLLKDKEGNVIE
SWMSTTEARVFEQKLIAGETYTLTEVTAPSGYEVAADITFTVNKDGTVS
VDGKAVD;
(amino acid sequence of CpCatcher)
SEQ ID NO: 7
NNQKSEVLKLNIIDDLPKEVLFSKHDIAGNELAGATIELSNKADGKVID
TWVSDGQGAHTFNLKPGNYVFTEKAAPEGYEVATAINFTVNPDGTVTSD
DV;
(amino acid sequence of RSCatcher);
SEQ ID NO: 8
TFTIDKTGKVLVNGEDVNGQVTMYDAALGDVVISKRAVNGTEELEGASL
KITDADGKTVAEWVSDSTPKTIQLDAGTYTLTEETAPDGYTVAESIEFV
VDAAG;
(amino acid sequence of ScoCatcher);
SEQ ID NO: 9
TVEDTSEVQKVEMKDDVTKVQISKTDISGKELPGAKLTILDKDGKTVES
WTSEEKPHYIEMLPIGEYTLREETAPDGYLVAEDVKFTVKDTGEIQKVV
MKDEVKPTATPT;
(amino acid sequence of SpnCatcher);
SEQ ID NO: 10
GNTIVMVDKLKELPPPPSTPPTKVKFSKKALTENGEELKGATIRLTKED
GSLVEEWVTDGTVKEFELKDGKYTFTEISAPAKYQVATAITFEVKNGKA
IVKGIEV;
SEQ ID NO: 11 (amino acid sequence of GvTag):
NTIVMVDKLKEVPTP;
SEQ ID NO: 12 (amino acid sequence of GbTag):
TNIEFVDGNKPHK;
SEQ ID NO: 13 (amino acid sequence of PsTag):
NTIVMVDKLKKTP;
SEQ ID NO: 14 (amino acid sequence of SaTag):
NTIVMVDKLKELPPP;
SEQ ID NO: 15 (amino acid sequence of SdTag):
DPIVMIDNDKPIT;
SEQ ID NO: 16 (amino acid sequence of AhTag):
NEIVMKDETTPVG;
SEQ ID NO: 17 (amino acid sequence of CpTag):
NHVVMVDGYAPKE;
SEQ ID NO: 18 (amino acid sequence of RSTag):
DQIVMVDVAKTTT;
SEQ ID NO: 19 (amino acid sequence of ScoTag):
QKVVMKDEVKPTA;
and
SEQ ID NO: 20 (amino acid sequence of SpnTag):
NTIVMVDKLKELP.

2. Construction of a Catcher-HPF fusion protein clone, a Tag-GFP fusion protein clone, and a recombinant vector thereof

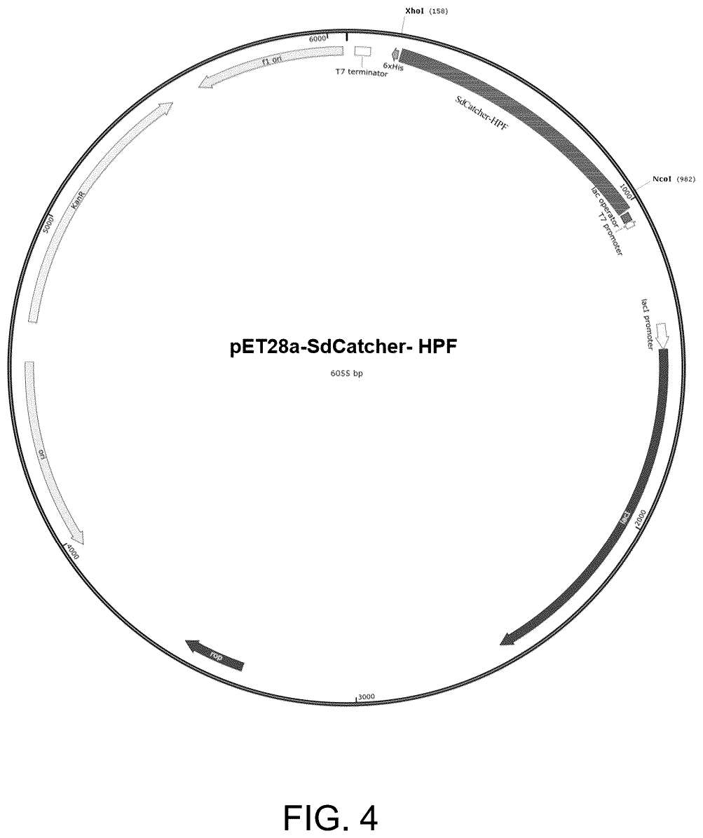

The above 10 different Catcher proteins and Helicobacter pylori _Ferritin (HPF) were used to construct 10 types of Catcher-HPF fusion proteins (amino acid sequences are shown in SEQ ID NOs: 21-30) according to the schematic diagram of FIG. 3; the nucleotide sequence encoding each Catcher-HPF fusion protein was cloned between restriction enzyme cutting sites Nco I and Xho I of an expression vector pET28a, to obtain a recombinant vector. The expression vector pET28a has a 6xHis Tag and a termination codon at downstream of the restriction enzyme cutting site Xho I. One of the expression vectors pET28a-SdCatcher-HPF is shown in FIG. 4.

SEQ ID NO: 21 (amino acid sequence of the fusion protein GvCatcher-HPF):
MVDKLKEVPTSTKVKFSKKALTENGEDLKGATIQLTKADGSLVKKWVTDGTV
TEFELKDGKYTFTETSAPAKYQVATAITFEVKNGKAIVKGIAVTGNTIVGSGDIIKLL
NEQVNKEMQSSNLYMSMSSWCYTHSLDGAGLFLFDHAAEEYEHAKKLIIFLNENNV
PVQLTSISAPEHKFEGLTQIFQKAYEHEQHISESINNIVDHAIKSKDHATFNFLQWYV
AEQHEEEVLFKDILDKIELIGNENHGLYLADQYVKGIAKSRKS;
SEQ ID NO: 22 (amino acid sequence of the fusion protein GbCatcher-HPF):
VVTDGYSSHDINISKADIDGNEIAGAKIVLTDKAGKQIDSWTSTKELHKVSLKP
GTYIFKETLAPEGFEVVTDITFTVNVDGTITVNDKQAKVNNDGVLGSGDIIKLLNEQ
VNKEMQSSNLYMSMSSWCYTHSLDGAGLFLFDHAAEEYEHAKKLIIFLNENNVPVQ
LTSISAPEHKFEGLTQIFQKAYEHEQHISESINNIVDHAIKSKDHATFNFLQWYVAEQ
HEEEVLFKDILDKIELIGNENHGLYLADQYVKGIAKSRKS;
SEQ ID NO: 23 (amino acid sequence of the fusion protein PsCatcher-HPF):
EKTKVKFSKKALTANGEELKGATIKLTKENGTVIKEWVTDGKLTEFELEDGSY
TFTEISAPDKYQVATAITFEVKAGKVLVTGTEVKGNTIVGSGDIIKLLNEQVNKEMQ
SSNLYMSMSSWCYTHSLDGAGLFLFDHAAEEYEHAKKLIIFLNENNVPVQLTSISAP
EHKFEGLTQIFQKAYEHEQHISESINNIVDHAIKSKDHATFNFLQWYVAEQHEEEVLF
KDILDKIELIGNENHGLYLADQYVKGIAKSRKS;
SEQ ID NO: 24 (amino acid sequence of the fusion protein SaCatcher-HPF):
STPSTKVKFSKKALTENGEELKGATIRLTKEDGSLVKEWVTDGTVKEFELKDG
KYTFTEISAPDKYQVATAITFEVKNGKVIVKGIEVTGNTIVGSGDIIKLLNEQVNKEM
QSSNLYMSMSSWCYTHSLDGAGLFLFDHAAEEYEHAKKLIIFLNENNVPVQLTSISA
PEHKFEGLTQIFQKAYEHEQHISESINNIVDHAIKSKDHATFNFLQWYVAEQHEEEVL
FKDILDKIELIGNENHGLYLADQYVKGIAKSRKS;
SEQ ID NO: 25 (amino acid sequence of the fusion protein SdCatcher-HPF):
SGETGQSGNTTIEEDSTTHVKFSKRDINGKELAGAMIELRNLSGQTIQSWVSDG
TVKDFYLMPGTYQFVETAAPEGYELAAPITFTIDEKGQIWVDSTLIVGDDPIGSGDIIK
LLNEQVNKEMQSSNLYMSMSSWCYTHSLDGAGLFLFDHAAEEYEHAKKLIIFLNEN
NVPVQLTSISAPEHKFEGLTQIFQKAYEHEQHISESINNIVDHAIKSKDHATFNFLQW
YVAEQHEEEVLFKDILDKIELIGNENHGLYLADQYVKGIAKSRKS;
SEQ ID NO: 26 (amino acid sequence of the fusion protein AhCatcher-HPF):
ITDKETGEDIYLVKDDVTRVSVKKMDITGQKEVAGARLLLKDKEGNVIESWM
STTEARVFEQKLIAGETYTLTEVTAPSGYEVAADITFTVNKDGTVSVDGKAVDGSGD
IIKLLNEQVNKEMQSSNLYMSMSSWCYTHSLDGAGLFLFDHAAEEYEHAKKLIIFLN
ENNVPVQLTSISAPEHKFEGLTQIFQKAYEHEQHISESINNIVDHAIKSKDHATFNFLQ
WYVAEQHEEEVLFKDILDKIELIGNENHGLYLADQYVKGIAKSRKS;
SEQ ID NO: 27 (amino acid sequence of the fusion protein CpCatcher-HPF):
NNQKSEVLKLNIIDDLPKEVLFSKHDIAGNELAGATIELSNKADGKVIDTWVSD
GQGAHTFNLKPGNYVFTEKAAPEGYEVATAINFTVNPDGTVTSDDVGSGDIIKLLNE
QVNKEMQSSNLYMSMSSWCYTHSLDGAGLFLFDHAAEEYEHAKKLIIFLNENNVPV
QLTSISAPEHKFEGLTQIFQKAYEHEQHISESINNIVDHAIKSKDHATFNFLQWYVAE
QHEEEVLFKDILDKIELIGNENHGLYLADQYVKGIAKSRKS;
SEQ ID NO: 28 (amino acid sequence of the fusion protein RSCatcher-HPF):
TFTIDKTGKVLVNGEDVNGQVTMYDAALGDVVISKRAVNGTEELEGASLKIT
DADGKTVAEWVSDSTPKTIQLDAGTYTLTEETAPDGYTVAESIEFVVDAAGGSGDII
KLLNEQVNKEMQSSNLYMSMSSWCYTHSLDGAGLFLFDHAAEEYEHAKKLIIFLNE
NNVPVQLTSISAPEHKFEGLTQIFQKAYEHEQHISESINNIVDHAIKSKDHATFNFLQ
WYVAEQHEEEVLFKDILDKIELIGNENHGLYLADQYVKGIAKSRKS;
SEQ ID NO: 29 (amino acid sequence of the fusion protein ScoCatcher-HPF):
TVEDTSEVQKVEMKDDVTKVQISKTDISGKELPGAKLTILDKDGKTVESWTSE
EKPHYIEMLPIGEYTLREETAPDGYLVAEDVKFTVKDTGEIQKVVMKDEVKPTATPT
GSGDIIKLLNEQVNKEMQSSNLYMSMSSWCYTHSLDGAGLFLFDHAAEEYEHAKKL
IIFLNENNVPVQLTSISAPEHKFEGLTQIFQKAYEHEQHISESINNIVDHAIKSKDHATF
NFLQWYVAEQHEEEVLFKDILDKIELIGNENHGLYLADQYVKGIAKSRKS;
SEQ ID NO: 30 (amino acid sequence of the fusion protein SpnCatcher-HPF):
GNTIVMVDKLKELPPPPSTPPTKVKFSKKALTENGEELKGATIRLTKEDGSLVE
EWVTDGTVKEFELKDGKYTFTEISAPAKYQVATAITFEVKNGKAIVKGIEVGSGDIIK
LLNEQVNKEMQSSNLYMSMSSWCYTHSLDGAGLFLFDHAAEEYEHAKKLIIFLNEN
NVPVQLTSISAPEHKFEGLTQIFQKAYEHEQHISESINNIVDHAIKSKDHATFNFLQW
YVAEQHEEEVLFKDILDKIELIGNENHGLYLADQYVKGIAKSRKS.

The 10 different Tag proteins and green fluorescent protein (GFP) were used to construct 10 types of Tag-GFP fusion protein clones (amino acid sequences are shown in

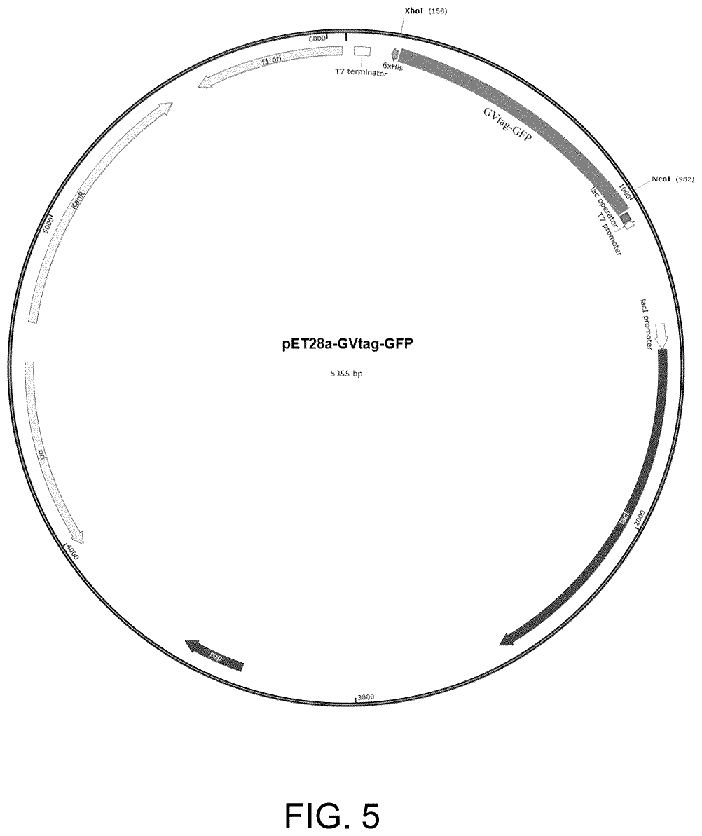

SEQ ID NOs: 31-40) according to the schematic diagram of FIG. 3; the nucleotide sequence encoding each Tag-GFP fusion protein was cloned between restriction enzyme cutting sites Nco I and Xho I of an expression vector pET28a, to obtain a recombinant plasmid. One of the expression vectors pET28a-GvTag-GFP is shown in FIG. 5.

SEQ ID NO: 31 (amino acid sequence of the fusion protein GvTag-GFP):
NTIVMVDKLKEVPTPGSGMVSKGEELFTGVVPILVELDGDVNGHKFSVSGEGE
GDATYGKLTLKFICTTGKLPVPWPTLVTTLTYGVQCFSRYPDHMKQHDFFKSAMPE
GYVQERTIFFKDDGNYKTRAEVKFEGDTLVNRIELKGIDFKEDGNILGHKLEYNYNS
HNVYIMADKQKNGIKVNFKIRHNIEDGSVQLADHYQQNTPIGDGPVLLPDNHYLST
QSALSKDPNEKRDHMVLLEFVTAAGITLGMDELYK;
SEQ ID NO: 32 (amino acid sequence of the fusion protein GbTag-GFP):
TNIEFVDGNKPHKGSGMVSKGEELFTGVVPILVELDGDVNGHKFSVSGEGEGD
ATYGKLTLKFICTTGKLPVPWPTLVTTLTYGVQCFSRYPDHMKQHDFFKSAMPEGY
VQERTIFFKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNHYLSTQSA
LSKDPNEKRDHMVLLEFVTAAGITLGMDELYK;
SEQ ID NO: 33 (amino acid sequence of the fusion protein PsTag-GFP):
NTIVMVDKLKKTPGSGMVSKGEELFTGVVPILVELDGDVNGHKFSVSGEGEG
DATYGKLTLKFICTTGKLPVPWPTLVTTLTYGVQCFSRYPDHMKQHDFFKSAMPEG
YVQERTIFFKDDGNYKTRAEVKFEGDTLVNRIELKGIDFKEDGNILGHKLEYNYNSH
NVYIMADKQKNGIKVNFKIRHNIEDGSVQLADHYQQNTPIGDGPVLLPDNHYLSTQS
ALSKDPNEKRDHMVLLEFVTAAGITLGMDELYK;
SEQ ID NO: 34 (amino acid sequence of the fusion protein SaTag-GFP):
NTIVMVDKLKELPPPGSGMVSKGEELFTGVVPILVELDGDVNGHKFSVSGEGE
GDATYGKLTLKFICTTGKLPVPWPTLVTTLTYGVQCFSRYPDHMKQHDFFKSAMPE
GYVQERTIFFKDDGNYKTRAEVKFEGDTLVNRIELKGIDFKEDGNILGHKLEYNYNS
HNVYIMADKQKNGIKVNFKIRHNIEDGSVQLADHYQQNTPIGDGPVLLPDNHYLST
QSALSKDPNEKRDHMVLLEFVTAAGITLGMDELYK;
SEQ ID NO: 35 (amino acid sequence of the fusion protein SdTag-GFP):
DPIVMIDNDKPITGSGMVSKGEELFTGVVPILVELDGDVNGHKFSVSGEGEGD
ATYGKLTLKFICTTGKLPVPWPTLVTTLTYGVQCFSRYPDHMKQHDFFKSAMPEGY
VQERTIFFKDDGNYKTRAEVKFEGDTLVNRIELKGIDFKEDGNILGHKLEYNYNSHN
VYIMADKQKNGIKVNFKIRHNIEDGSVQLADHYQQNTPIGDGPVLLPDNHYLSTQSA
LSKDPNEKRDHMVLLEFVTAAGITLGMDELYK.
SEQ ID NO: 36 (amino acid sequence of the fusion protein AhTag-GFP):
NEIVMKDETTPVGGSGMVSKGEELFTGVVPILVELDGDVNGHKFSVSGEGEGDATY
GKLTLKFICTTGKLPVPWPTLVTTLTYGVQCFSRYPDHMKQHDFFKSAMPEGYVQE
RTIFFKDDGNYKTRAEVKFEGDTLVNRIELKGIDFKEDGNILGHKLEYNYNSHNVYI
MADKQKNGIKVNFKIRHNIEDGSVQLADHYQQNTPIGDGPVLLPDNHYLSTQSALS
KDPNEKRDHMVLLEFVTAAGITLGMDELYK;
SEQ ID NO: 37 (amino acid sequence of the fusion protein CpTag-GFP):
NHVVMVDGYAPKEGSGMVSKGEELFTGVVPILVELDGDVNGHKFSVSGEGEGDAT
YGKLTLKFICTTGKLPVPWPTLVTTLTYGVQCFSRYPDHMKQHDFFKSAMPEGYVQ
ERTIFFKDDGNYKTRAEVKFEGDTLVNRIELKGIDFKEDGNILGHKLEYNYNSHNVY
IMADKQKNGIKVNFKIRHNIEDGSVQLADHYQQNTPIGDGPVLLPDNHYLSTQSALS
KDPNEKRDHMVLLEFVTAAGITLGMDELYK;
SEQ ID NO: 38 (amino acid sequence of the fusion protein RSTag-GFP):
DQIVMVDVAKTTTGSGMVSKGEELFTGVVPILVELDGDVNGHKFSVSGEGEGDATY
GKLTLKFICTTGKLPVPWPTLVTTLTYGVQCFSRYPDHMKQHDFFKSAMPEGYVQE
RTIFFKDDGNYKTRAEVKFEGDTLVNRIELKGIDFKEDGNILGHKLEYNYNSHNVYI
MADKQKNGIKVNFKIRHNIEDGSVQLADHYQQNTPIGDGPVLLPDNHYLSTQSALS
KDPNEKRDHMVLLEFVTAAGITLGMDELYK;
SEQ ID NO: 39 (amino acid sequence of the fusion protein ScoTag-GFP):
QKVVMKDEVKPTAGSGMVSKGEELFTGVVPILVELDGDVNGHKFSVSGEGEGDAT
YGKLTLKFICTTGKLPVPWPTLVTTLTYGVQCFSRYPDHMKQHDFFKSAMPEGYVQ
ERTIFFKDDGNYKTRAEVKFEGDTLVNRIELKGIDFKEDGNILGHKLEYNYNSHNVY
IMADKQKNGIKVNFKIRHNIEDGSVQLADHYQQNTPIGDGPVLLPDNHYLSTQSALS
KDPNEKRDHMVLLEFVTAAGITLGMDELYK;
SEQ ID NO: 40 (amino acid sequence of the fusion protein SpnTag-GFP):
NTIVMVDKLKELPGSGMVSKGEELFTGVVPILVELDGDVNGHKFSVSGEGEGDATY
GKLTLKFICTTGKLPVPWPTLVTTLTYGVQCFSRYPDHMKQHDFFKSAMPEGYVQE
RTIFFKDDGNYKTRAEVKFEGDTLVNRIELKGIDFKEDGNILGHKLEYNYNSHNVYI
MADKQKNGIKVNFKIRHNIEDGSVQLADHYQQNTPIGDGPVLLPDNHYLSTQSALS
KDPNEKRDHMVLLEFVTAAGITLGMDELYK.

The specific sequence information is specifically shown in Table 1.

TABLE 1

| | | | Amino acid | Amino acid |
|---|---|---|---|---|
| | Amino acid | Amino acid | sequence of the | sequence of the |
| | sequence of | sequence of | fusion protein | fusion protein |
| | Catcher protein | Tag peptide | Catcher-HPF | Tag-GFP |
| Gardnerella | SEQ ID NO: 1 | SEQ ID NO: 11 | SEQ ID NO: 21 | SEQ ID NO: 31 |
| vaginalis (Gv) | | | | |
| Granulicatella | SEQ ID NO: 2 | SEQ ID NO: 12 | SEQ ID NO: 22 | SEQ ID NO: 32 |
| balaenopterae (Gb) | | | | |
| Peptostreptococcus | SEQ ID NO: 3 | SEQ ID NO: 13 | SEQ ID NO: 23 | SEQ ID NO: 33 |
| sp (Ps) | | | | |
| Streptococcus | SEQ ID NO: 4 | SEQ ID NO: 14 | SEQ ID NO: 24 | SEQ ID NO: 34 |
| anginosus subsp (Sa) | | | | |
| Streptococcus | SEQ ID NO: 5 | SEQ ID NO: 15 | SEQ ID NO: 25 | SEQ ID NO: 35 |
| dysgalactiae (Sd) | | | | |
| Anaerobutyricum | SEQ ID NO: 6 | SEQ ID NO: 16 | SEQ ID NO: 26 | SEQ ID NO: 36 |
| hallii (Ah) | | | | |
| Clostridium | SEQ ID NO: 7 | SEQ ID NO: 17 | SEQ ID NO: 27 | SEQ ID NO: 37 |
| perfringens (Cp) | | | | |
| Ruminococcus sp | SEQ ID NO: 8 | SEQ ID NO: 18 | SEQ ID NO: 28 | SEQ ID NO: 38 |
| (Rs) | | | | |
| Streptococcus | SEQ ID NO: 9 | SEQ ID NO: 19 | SEQ ID NO: 29 | SEQ ID NO: 39 |
| constellatus (Sco) | | | | |
| Streptococcus | SEQ ID NO: 10 | SEQ ID NO: 20 | SEQ ID NO: 30 | SEQ ID NO: 40 |
| pneumoniae (Spn) | | | | |

3. Expression of the fusion protein Catcher-HPF and the fusion protein Tag-GFP

The prepared 20 recombinant plasmids were transformed into DH5a competent cells, and cultured overnight at 37° C.; positive clones were screened out and PCR-identified. The positive clones were sent for sequencing, and the plasmids was extracted after the sequencing result was correct.

The plasmids with correct sequencing of the 20 recombinant plasmids were transfected into BL21(DE3) to obtain recombinant strains, respectively for the expression of a prokaryotic protein; the recombinant strains were subjected to enlarged culture with kanamycin-resistant LB media at 37° C. and 220 rpm/min until 0D600 was about 0.6 (about 4 h), and then IPTG with a final concentration of 1 mM was added to induce protein expression at 16° C. and 220 rpm/min. 18 h later after the induction, bacterial cells were dissolved and disrupted ultrasonically; then the solution was centrifuged and precipitate was discarded.

Supernatant was incubated with Ni-NTA agarose (GE Healthcare) to enrich His-labeled Catcher-HPF and Tag-GFP, and then the proteins were eluted by a Tris buffer solution containing imidazole. The purified proteins were concentrated and replaced by 20 mM Tri-HCl+50 mM NaCl buffer solution (pH=7.5). The proteins were subjected to concentration determination by BCA assay and stained by Coomassie blue, to detect the expression of each protein. Anaerobutyricum hallii (Ah), Clostridium perfringens (Cp), Ruminococcus sp (Rs), Streptococcus constellatus (Sco) and Streptococcus pneumoniae (Spn).

4. Incubation of the Tag-GFP and the Catcher-HPF

Each purified Tag-GFP and each purified Catcher-HPF were incubated, respectively. Incubation of the Tag-GFP and the Catcher-HPF in a 20 mM Tri-HCl+50 mM NaCl Tris buffer solution (pH=7.5) according to a molar ratio of 1:1, i.e., 5 nmol of the Tag-GFP and 5 nmol of the Catcher-HPF were put to 1 ml of the buffer solution and incubated for 1 h at room temperature.

### II. Experimental Results

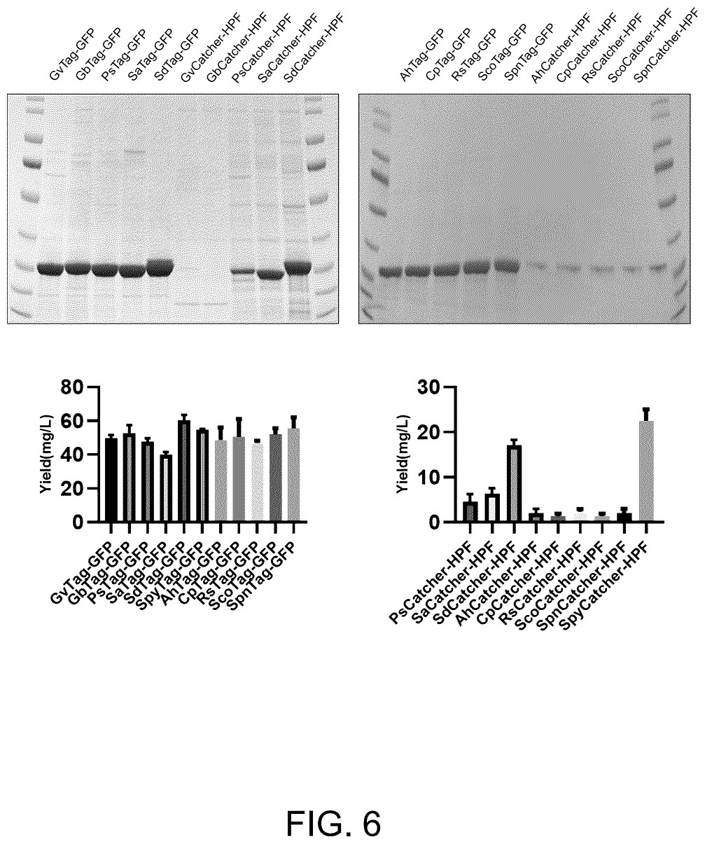

Coomassie blue staining results are shown in FIG. 6. Ps, Sa and Sd had high expression quantity of Catcher proteins, and these Catcher proteins were incubated with the Tag-GFP from the above five bacteria in a way of permutation and combination.

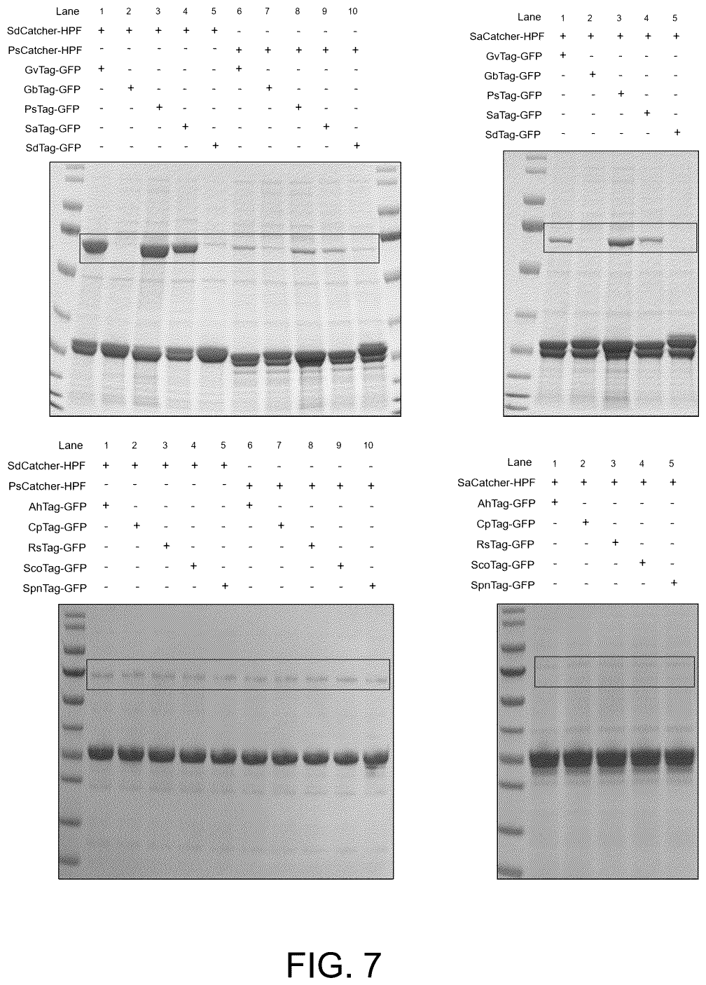

Incubation results are shown in FIG. 7. After Catcher-HPF and Tag-GFP were bound together, the size of the protein was 65 KDa. The results show the three combinations: the SdCatcher protein of Sd with GvTag-GFP (SEQ ID NO: 31), PsTag-GFP (SEQ ID NO: 33) and SaTag-GFP (SEQ ID NO: 34) have relatively effective binding capacity. Thereby three combined binding systems, namely, GvTag/SdCatcher (SEQ ID NO: 11 and SEQ ID NO: 5),

PsTag/SdCatcher (SEQ ID NO: 13 and SEQ ID NO: 5) and SaTag/SdCatcher (SEQ ID NO: 14 and SEQ ID NO: 5) were screened out.

### EXAMPLE 3 USE OF THESE THREE COMBINED BINDING SYSTEMS TO AN RBD SUBUNIT NANOVACCINE SYSTEM OF THE S PROTEIN OF SARS-COV-2 NOVEL CORONAVIRUS

### I. Experimental method

These three combined binding systems of GvTag/SdCatcher, PsTag/SdCatcher, and SaTag/SdCatcher screened in Example 2 were then applied to an RBD subunit nanovaccine system of the S protein of SARS-CoV-2 novel coronavirus.

1. Construction of recombinant plasmid

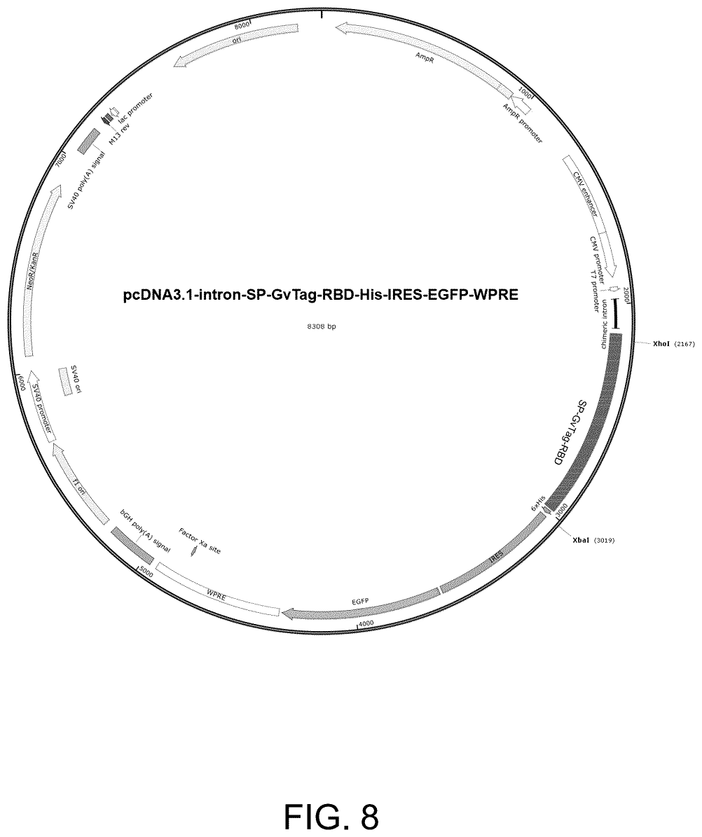

Three Tag-RBD fusion protein clones were constructed by GvTag, PsTag, and SaTag with RBD, respectively according to the schematic diagram as shown in FIG. 3. A coding sequence for a secreting peptide SP was added at 5′ terminal of the Tag-RBD nucleotide sequence; Tag and RBD were spaced by a Linker GSG; 6xHis and translation termination codon were added at 3′ terminal thereof, so as to obtain SP-GvTag-RBD-His, SP-PsTag-RBD-His and SP-SaTag-RBD-His (amino acid sequences encoded are shown in SEQ ID NOs: 41-43), respectively. SP-GvTag-RBD-His, SP-PsTag-RBD-His and SP-SaTag-RBD-His were then cloned between the restriction enzyme cutting sites Xho I and Xba I of an expression vector (pcDNA3.1-Intron-WPRE) (as shown in FIG. 8) to which Intron and WPRE were added for expression enhancement, thereby constructing an expression vector pcDNA3.1-Intron-SP-Tag-RBD-IRES-GFP-WPRE. One of the expression vectors pcDNA3.1-Intron-SP-GvTag-RBD-His-IRES-GFP-WPRE is shown in FIG. 8.

SEQ ID NO: 41 (amino acid sequence of the fusion protein SP-GvTag-RBD-His):
MGILPSPGMP
ALLSLVSLLSVLLMGCVAGSGNTIVMVDKLKEVPTPGSGRVQPTESIVRF
PNITNLCPFG
EVFNATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLD
SKVGGNYNYLYRLFRKSNLKPFERDISTEIYQAGSTPCNGVEGENCYFPLQSYGFQPT
NGVGYQPYRVVVLSFELLH APATVCGPKK STNLVKNKCV NFHHHHHH;
SEQ ID NO: 42 (amino acid sequence of the fusion protein SP-PsTag-RBD-His):
MGILPSPGMPALLSLVSLLSVLLMGCVAGSGNTIVMVDKLKKTPGSGRVQPTE
SIVRFPNITNLCPFGEVFNATRFASVYAWNRKRISNCVADYSVLYNSASFSTFKCYGV
SPTKLNDLCFTNVYADSFVIRGDEVRQIAPGQTGKIADYNYKLPDDFTGCVIAWNSN
NLDSKVGGNYNYLYRLFRKSNLKPFERDISTEIYQAGSTPCNGVEGENCYFPLQSYG
FQPTNGVGYQPYRVVVLSFELLHAPATVCGPKKSTNLVKNKCVNFHHHHHH;
SEQ ID NO: 43 (amino acid sequence of the fusion protein SP-SaTag-RBD-His):
MGILPSPGMPALLSLVSLLSVLLMGCVAGSGNTIVMVDKLKELPPPGSGRVQP
TESIVRFPNITNLCPFGEVFNATRFASVYAWNRKRISNCVADYSVLYNSASFSTFKCY
GVSPTKLNDLCFTNVYADSFVIRGDEVRQIAPGQTGKIADYNYKLPDDFTGCVIAWN
SNNLDSKVGGNYNYLYRLFRKSNLKPFERDISTEIYQAGSTPCNGVEGENCYFPLQS
YGFQPTNGVGYQPYRVVVLSFELLHAPATVCGPKKSTNLVKNKCVNFHHHHHH.

2. Expression and purification of the recombinant protein

The prepared recombinant plasmids were transformed into DH5a competent cells, and cultured overnight at 37° C.; positive clones were screened and PCR-identified. Deendotoxin-plasmids were extracted, and transfected into HEK293F cells in a way of lipofection transfection, 5 days alter after the transfection, the cellular supernatant was harvested after centrifuge, and then the interest protein was purified.

The supernatant was incubated with Ni-NTA agarose (GE Healthcare); His-labeled Tag-RBD was enriched and then protein elution was performed by a Tris buffer solution containing imidazole. The purified proteins were concentrated and replaced by 20 mM Tri-HCl+50 mM NaCl buffer solution (pH=7.5). The proteins were subjected to concentration determination by BCA and stained by Coomassie blue.

3. Self-assembly of Ferritin of a tetracosemer nanoparticle

5 nmol of the purified Tag-RBD protein was incubated with 5 nmol of the SdCatcher-HPF protein prokaryotically expressed and purified in Example 3 in 1 ml of buffer system (20 mM Tri-HCl+50 mM NaCl, pH=7.5) for 1 h at room temperature. After the Tag-RBD and the SdCatcher-HPF were bound together, the monomeric protein had a size of 70 KDa.

### II. Experimental results

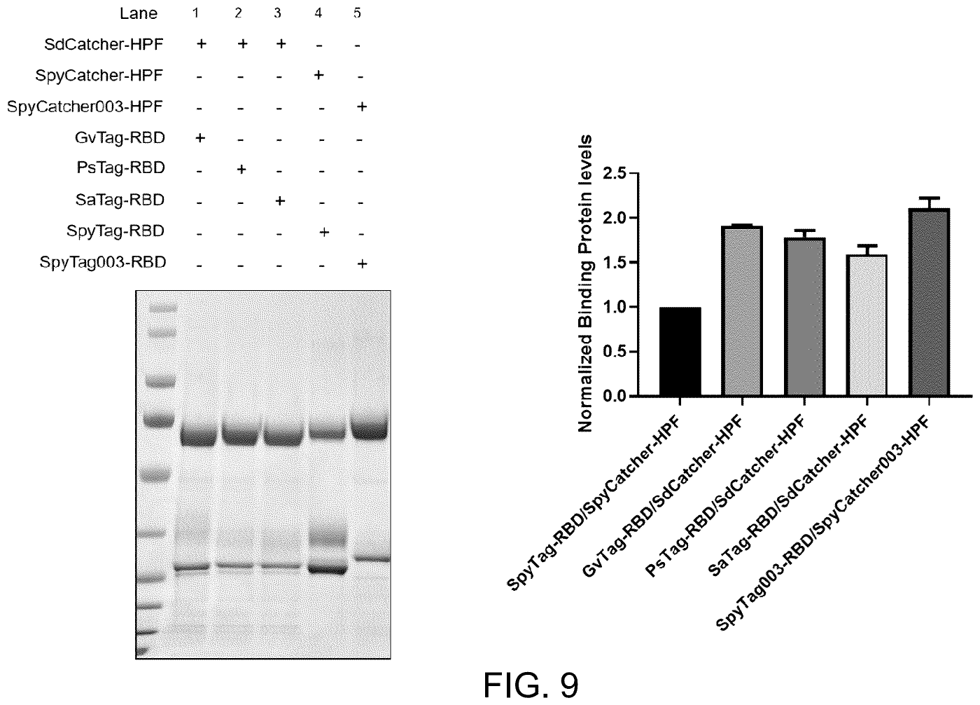

Results are as shown in FIG. 9. RBD was self-assembled to Ferritin of a spherical tetracosemer nanoparticle. The use of GvTag/SdCatcher, PsTag/SdCatcher, and SaTag/SdCatcher has effectively improved binding efficiency compared to the existing Spy Tag/SpyCatcher system. Moreover, the protein binding efficiency is optimal when using the GvTag/SdCatcher system, but inferior to the optimized SpyTag003/SpyCatcher003 system.

### EXAMPLE 4 USE OF THE OPTIMIZED GVTAG/SDCATCHER SYSTEM TO THE RBD SUBUNIT NANOVACCINE SYSTEM OF THE S PROTEIN OF SARS-COV-2 NOVEL CORONAVIRUS

### I. Experimental method

Three amino acids RVG were added at N terminal of the GvTag sequence to construct a peptide tag GvTag001 with 18 amino acids (amino acid sequence is shown in SEQ ID NO: 44: RVGNTIVMVDKLKEVPTP); three amino acids KKVG were added at N terminal of the GvTag sequence to construct a peptide tag GvTag002 with 19 amino acids (amino acid sequence is shown in SEQ ID NO: 45: KKVGNTIVMVDKLKEVPTP); three amino acids KVG were added at N terminal of the GvTag sequence to construct a peptide tag GvTagOpti with 18 amino acids (amino acid sequence is shown in SEQ ID NO: 46: KVGNTIVMVDKLKEVPTP); namely, 3 artificially modified GvTags (amino acid sequences are shown in SEQ ID NOs: 44-46, respectively) were obtained.

The 3 artificially modified GvTags (amino acid sequences are shown in SEQ ID NOs: 44-46, respectively) and RBD served to construct fusion protein clones GvTag001-RBD, GvTag002-RBD and GvTagOpti-RBD, respectively according to the schematic diagram as shown in FIG. 3. A coding sequence for a secreting peptide SP was added at each 5′ terminal of the nucleotide sequence of GvTag001-RBD, GvTag002-RBD and GvTagOpti-RBD; and a translation termination codon was added at each 3′ terminal thereof, so as to obtain SP-GvTag001-RBD-His, SP-GvTag002-RBD-His and SP-GvTagOpti-RBD-His. The amino acid sequences encoded thereby are shown in SEQ ID NOs: 47-49, respectively. SP-GvTag001-RBD-His, SP-GvTag002-RBD-His and SP-GvTagOpti-RBD-His were then cloned between the restriction enzyme cutting sites Xho I and Xba I of an expression vector (pcDNA3.1-Intron-WPRE) to which Intron and WPRE were added for expression enhancement, thereby constructing expression vectors pcDNA3.1-Intron-SP-GvTag001-RBD-His-IRES-GFP-WPR, pcDNA3.1-Intron-SP-GvTag002-RBD-His-IRES-GFP-WPR, and pcDNA3.1-Intron-SP-GvTagOpti-RBD-His-IRES-GFP-WPRE, respectively.

SEQ ID NO: 47 (amino acid sequence of the fusion protein Sp-GvTag001-RBD-His):
MGILPSPGMPALLSLVSLLSVLLMGCVAGSGRVGNTIVMVDKLKEVPTPGSGR
VQPTESIVRFPNITNLCPFGEVFNATRFASVYAWNRKRISNCVADYSVLYNSASFSTF
KCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGENCYFP
LQSYGFQPTNGVGYQPYRVVVLSFELLHAPATVCGPKKSTNLVKNKCVNFHHHHH
H;
SEQ ID NO: 48 (amino acid sequence of the fusion protein Sp-GvTag002-RBD-His):
MGILPSPGMPALLSLVSLLSVLLMGCVAGSGKKVGNTIVMVDKLKEVPTPGSG
RVQPTESIVRFPNITNLCPFGEVFNATRFASVYAWNRKRISNCVADYSVLYNSASFST
FKCYGVSPTKLNDLCFTNVYADSFVIRGDEVRQIAPGQTGKIADYNYKLPDDFTGCV
IAWNSNNLDSKVGGNYNYLYRLFRKSNLKPFERDISTEIYQAGSTPCNGVEGENCYF
PLQSYGFQPTNGVGYQPYRVVVLSFELLHAPATVCGPKKSTNLVKNKCVNFHHHHH
H;
SEQ ID NO.: 49 (amino acid sequence of the fusion protein Sp-GvTagOpti-RBD-His):
MGILPSPGMPALLSLVSLLSVLLMGCVAGSGKVGNTIVMVDKLKEVPTPGSGR
VQPTESIVRFPNITNLCPFGEVFNATRFASVYAWNRKRISNCVADYSVLYNSASFSTF
KCYGVSPTKLNDLCFTNVYADSFVIRGDEVRQIAPGQTGKIADYNYKLPDDFTGCVI
AWNSNNLDSKVGGNYNYLYRLFRKSNLKPFERDISTEIYQAGSTPCNGVEGFNCYFP
LQSYGFQPTNGVGYQPYRVVVLSFELLHAPATVCGPKKSTNLVKNKCVNFHHHHH
H.

The GvTag001-RBD, GvTag002-RBD, and GvTagOpti-RBD proteins were purified by the method of Example 3, respectively. 5 nmol of the purified GvTag001-RBD, GvTag002-RBD and GvTagOpti-RBD were incubated with 5 nmol of the SdCatcher-HPF protein prokaryotically expressed in Example 3 in 1 ml of buffer system (20 mM Tri-HC1 +50 mM NaCl, pH=7.5) for lh at room temperature.

### II. Experimental results

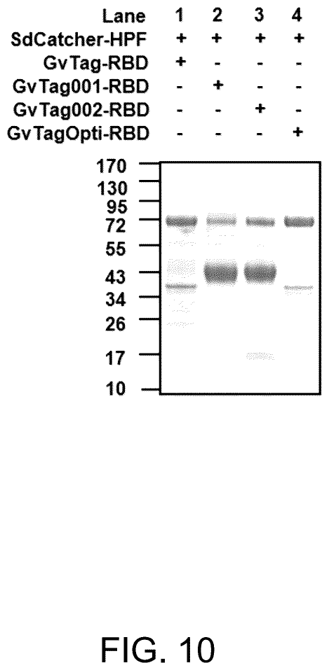

The results are shown in FIG. 10. The amount of the GvTag003-RBD-SdCatcher-HPF polymeric antigen proteins obtained using the GvTagOpti/SdCatcher (SEQ ID NO: 46 and SEQ ID NO: 5) system in this example is more than that of the polymeric antigen proteins obtained using the GvTag/SdCatcher system in Example 3, the GvTag001/SdCatcher (SEQ ID NO: 44 and SEQ ID NO: 5) system in this example, and the GvTag002/SdCatcher (SEQ ID NO: 44 and SEQ ID NO: 5) system in this example.

### EXAMPLE 5 COMPARISON OF THE BINDING EFFICIENCY BETWEEN THE GVTAGOPTI/SDCATCHER SYSTEM AND THE SPYTAG003/SPYCATCHER003 SYSTEM

### I. Experimental method

5 nmol of the GvTagOpti-RBD protein obtained in Example 4 was bound to 5 nmol of the SdCatcher-HPF obtained in Example 3, and then diluted by a 20 mM Tri-HCl+50 mM NaCl buffer solution (pH=7.5) at the dilution ratio of 1:2, 1:5 and 1:10. The specific operating steps were as follows: 5 nmol of the GvTagOpti-RBD protein obtained in Example 4 was incubated with 5 nmol of the SdCatcher-HPF obtained in Example 3 in 1 ml of a buffer system (20 mM Tri-HC1 +50 mM NaCl) at room temperature for lh to obtain a solution A; 50 μl of the solution A was then added to 50 μl of 20 mM Tri-HC1 +50 mM NaCl buffer solution (diluted at 1:2) to obtain a solution B. 40 μl of the solution B was added to 60 μl of 20 mM Tri-HCl+50 mM NaCl buffer solution (diluted at 1:5) to obtain a solution C. 50 μl of the solution C was then added to 50 μl of 20 mM Tri-HCl+50 mM NaCl buffer solution (diluted at 1:10) to obtain a solution D. 20 μl of the solution B, 20 μl of the solution C, and 20 μl of the solution D were then pipetted into three 1.5 ml EP tubes, respectively, and 5 μl of 5×loading was added respectively to each tube for protein boiling at 100° C. After being boiled for 5 min, 20 μl of each sample was subjected to protein electrophoresis and Coomassie brilliant blue staining.

Similarly, 5 nmol of the SpyTag003-RBD protein was bound to 5 nmol of the SdCatcher003-HPF, and then diluted by a 20 mM Tri-HCl+50 mM NaCl buffer solution (pH=7.5) at the dilution ratio of 1:2, 1:5 and 1:10. The specific operating steps are described above. The diluted binding proteins were then compared in sequence.

### II. Experimental results

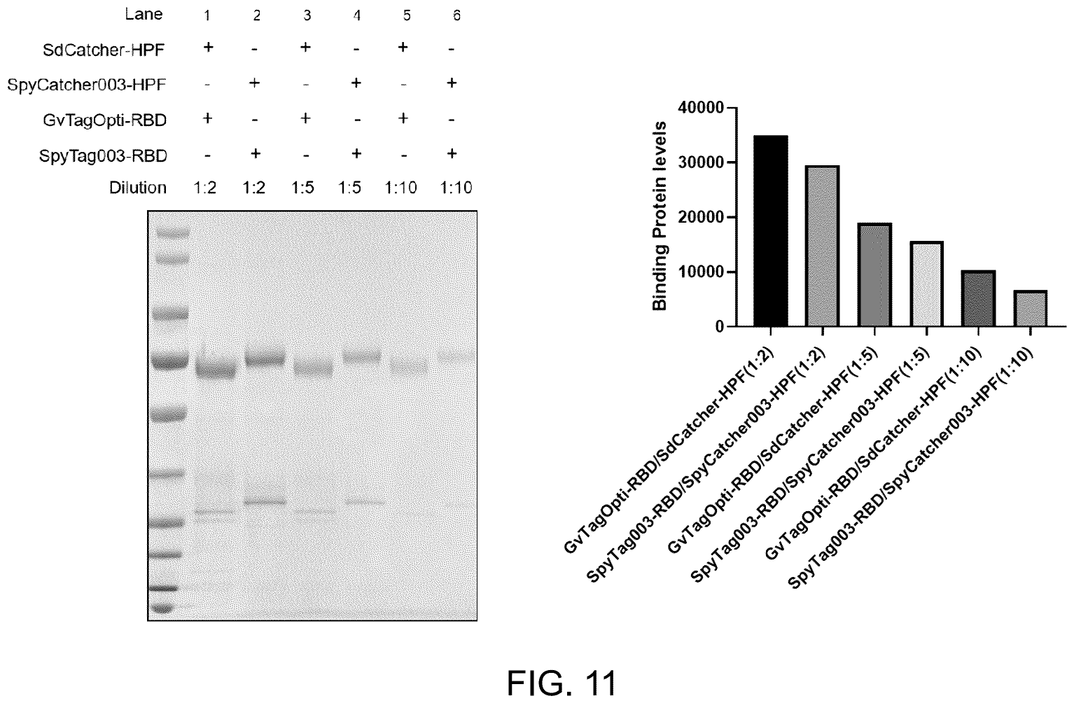

The results are shown in FIG. 11. In the RBD subunit nanovaccine system of SARS-CoV-2 novel coronavirus, the GvTagOpti/SdCatcher (SEQ ID NO: 46 and SEQ ID NO: 5) in Example 4 has strong binding efficiency than that of the SpyTag003/SpyCatcher003 system.

### Example 6

### I. Experimental method

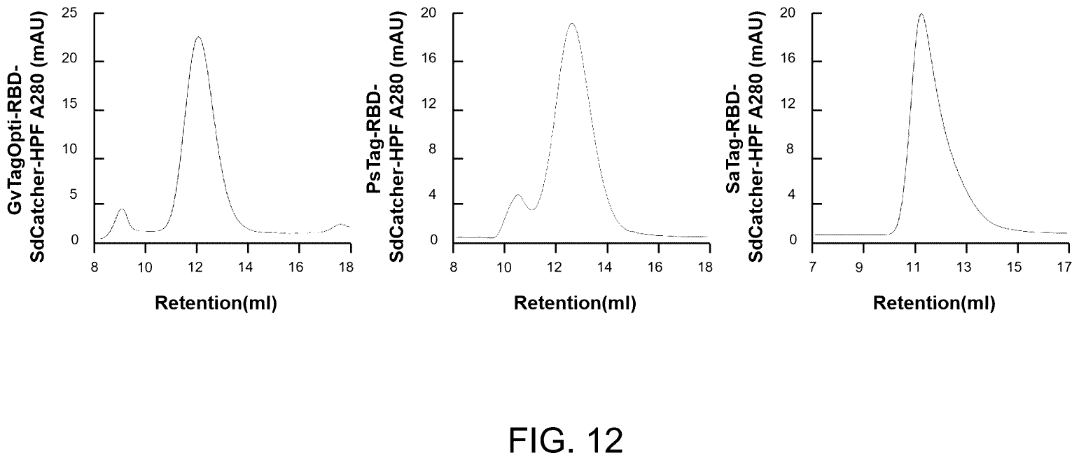

The purified GvTagOpti-RBD-SdCatcher-HPF polymeric antigen obtained from Example 4, the purified PsTag-RBD-SdCatcher-HPF polymeric antigen and SaTag-RBD-SdCatcher-HPF obtained from Example 3 were purified by molecular sieve chromatography with a Siperose6 Increase10/300 GL column (GE), to obtain the tetracosemer RBD-HPF proteins (as shown in FIG. 12). The buffer solution for molecular sieve chromatography was: 20 mM Tri-HCl+50 mM NaCl, pH=7.5. After being concentrated, the interest proteins were diluted with 20 mM Tri-HCl+50 mM NaCl (pH=7.5) to 100 μl according to Table 2, and then emulsified with an Alum adjuvant with same volume in groups.

TABLE 2

| | Antigen | | Quantity of |
|---|---|---|---|
| Antigen/Control | content | Adjuvant | animals (pcs.) |
| PBS | 0 | Alum | 5 |
| SpyTag003-RBD (monomer) | 0.2 nM | Alum | 5 |
| GvTagOpti-RBD-SdCatcher-HPF (Example 4) | 0.2 nM | Alum | 5 |
| PsTag-RBD-SdCatcher-HPF (Example 3) | 0.2 nM | Alum | 5 |
| SaTag-RBD-SdCatcher-HPF (Example 3) | 0.2 nM | Alum | 5 |
| SpyTag003-RBD-SpyCatcher003-HPF | 0.2 nM | Alum | 5 |

Balb/C mice aged at 6-8 weeks were then grouped and immunized. Each mouse was inoculated in a volume of 200 ial via subcutaneous injection. The mice were subjected to blood sampling from eye sockets on the 14th day. After standing for a period of time, the mice serum was separated out, centrifuged at 4° C. and 2800 rpm for 15 min, and then immediately used for Anti-RBD IgG ELISA test.

### 5 II. Experimental Results

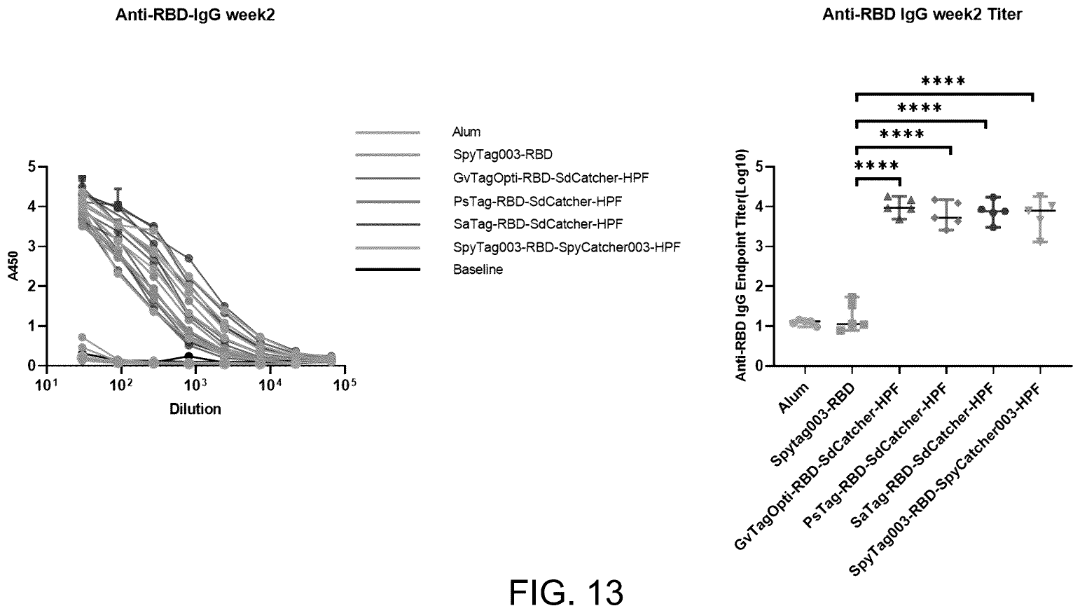

The results are shown in FIG. 13. In the GvTagOpti/SdCatcher system (SEQ ID NO: 46 and SEQ ID NO: 5), the PsTag/SdCatcher system (SEQ ID NO: 13 and SEQ ID NO: 5), the SaTag/SdCatcher system (SEQ ID NO: 14 and SEQ ID NO:5), and the SpyTag003/SpyCatcher003 system of the present disclosure, the RBD-HPF proteins linked using an isopeptide bond thereof were emulsified with the Alum adjuvant, respectively, and then immunized mice for once for 14 days, which may stimulate humoral immunity of the mice. Compared with the RBD monomer without the linking system using an isopeptide bond, the RBD-HPF proteins linked using the isopeptide bond of the GvTagOpti/SdCatcher system, the PsTag/SdCatcher system, the SaTag/SdCatcher system, and the SpyTag003/SpyCatcher003 system generated a high titer of Anti-RBD IgG, and had immunogenicity much higher than the RBD monomer.

Finally, it should be indicated that the above examples are merely used to specify the technical solutions of the present disclosure, but not construed as limiting the protection scope of the present disclosure. A person skilled in the art can further make other different forms of alterations or changes on the basis of the above description. Moreover, there is no need and no way to enumerate all the embodiments one by one. Any modification, equivalent substitution and improvement made within the spirit and principle of the present disclosure should fall within the protection scope of the claims of the present disclosure.

1. A combination of peptide linkers for protein covalent self-assembly using a spontaneous isopeptide bond, comprising a peptide linker 1 and a peptide linker 2; wherein the peptide linker 1 comprises a peptide chain having an amino acid sequence as shown in SEQ ID NO: 5, and the peptide linker 2 comprises any one or more of peptide chains having amino acid sequences as shown in any one or more of SEQ ID NOs: 11, 13, and 14.

2. The combination of peptide linkers according to claim 1, wherein the peptide linker 2 comprises a peptide chain having an amino acid sequence as shown in SEQ ID NO: 46.

3. A recombinant and synthetic peptide chain, comprising one or more of the peptide linker 1 and/or the peptide linker 2 in the combination according to claim 1.

4. A nucleic acid molecule, comprising a nucleotide sequence encoding one or more of the peptide linker 1 and/or the peptide linker 2 in the combination according to claim 1.

5. A vector, comprising the nucleic acid molecule according to claim 4.

6. A cell, comprising the nucleic acid molecule according to claim 4.

7. Use of the combination according to claim 1 in self-assembling two molecules or components using an isopeptide bond, wherein the two molecules or components are connected with the peptide linker 1 and the peptide linker 2 in the combination, respectively.

8. A method for self-assembling two molecules or components using an isopeptide bond, comprising:
providing a first molecule or a first component comprising the peptide linker 1 in the combination according to claim 1;
providing a second molecule or a second component comprising the peptide linker 2 in the combination; and
contacting the first molecule or the first component with the second molecule or the second component in a case of spontaneously forming an isopeptide bond between the peptide linker 1 and the peptide linker 2, such that the first molecule or the first component is self-assembled with the second molecule or the second component to form a complex using the isopeptide bond.

9. A kit for self-assembling two molecules or components using an isopeptide bond, comprising one of the peptide linker 1 and the peptide linker 2 in the combination according to claim 1, a nucleic acid molecule encoding the peptide linker 1 and the peptide linker 2 in the combination, or a cell comprising a vector encoding the peptide linker 1 and the peptide linker 2 in the combination.

10. Use of of the combination according to claim 1, in preparing a kit for self-assembling two molecules or components, or in self-assembling two molecules or components using an isopeptide bond.

11. A recombinant and synthetic peptide chain, comprising one or more of the peptide linker 1 and/or the peptide linker 2 in the combination according to claim 2.

12. A nucleic acid molecule, comprising a nucleotide sequence encoding one or more of the peptide linker 1 and/or the peptide linker 2 in the combination according to claim 2.

13. A vector, comprising the nucleic acid molecule according to claim 12.

14. A cell, comprising the vector according to claim 5.

15. Use of the combination according to claim 2 in self-assembling two molecules or components using an isopeptide bond, wherein the two molecules or components are connected with the peptide linker 1 and the peptide linker 2 in the combination, respectively.

16. A method for self-assembling two molecules or components using an isopeptide bond, comprising:
providing a first molecule or a first component comprising the peptide linker 1 in the combination according to claim 2;
providing a second molecule or a second component comprising the peptide linker 2 in the combination; and
contacting the first molecule or the first component with the second molecule or the second component in the a case of spontaneously forming an isopeptide bond between the peptide linker 1 and the peptide linker 2, such that the first molecule or the first component is self-assembled with the second molecule or the second component to form a complex using the isopeptide bond.

17. A kit for self-assembling two molecules or components using an isopeptide bond, comprising one of the peptide linker 1 and the peptide linker 2 in the combination according to claim 2, a nucleic acid molecule encoding the peptide linker 1 and the peptide linker 2 in the combination, or a cell comprising a vector encoding the peptide linker 1 and the peptide linker 2 in the combination.

18. Use of the nucleic acid molecule according to claim 3 in preparing a kit for self-assembling two molecules or components, or in self-assembling two molecules or components using an isopeptide bond.

19. Use of the vector according to claim 5 in preparing a kit for self-assembling two molecules or components, or in self-assembling two molecules or components using an isopeptide bond.

20. Use of the cell according to claim 6 in preparing a kit for self-assembling two molecules or components, or in self-assembling two molecules or components using an isopeptide bond.
